(19) United States
(12) Patent Application Publication
(10) Pub. No.: US 2024/0358996 A1
(43) Pub. Date: Oct. 31, 2024

(54) CIRCULATORY SUPPORT SYSTEM

(71) Applicant: Boston Scientific Scimed, Inc., Maple Grove, MN (US)

(72) Inventors: Qian Liu, Plymouth, MN (US); Aaron Chalekian, Minneapolis, MN (US); Brian R. Reynolds, Ramsey, MN (US)

(73) Assignee: Boston Scientific Scimed, Inc., Maple Grove, MN (US)

(21) Appl. No.: 18/647,009

(22) Filed: Apr. 26, 2024

Related U.S. Application Data
(60) Provisional application No. 63/499,107, filed on Apr. 28, 2023.

(51) Int. Cl. A61M 60/17; A61M 60/216; A61M 60/416; A61M 60/508; A61M 60/585; A61M 60/90

(52) U.S. Cl. CPC A61M 60/17; A61M 60/216; A61M 60/416; A61M 60/508; A61M 60/585; A61M 60/90; A61M 2205/04; A61M 2205/103; A61M 2205/3303; A61M 2205/3327; A61M 2205/3334; A61M 2205/502; A61M 2210/125; A61M 2210/127

(57) ABSTRACT

An example cardiac pump system includes a catheter shaft having a proximal end region coupled to a handle and a distal end region coupled to a cardiac pump, wherein the cardiac pump includes an impeller housing, a cannula and an impeller, wherein the cannula includes a distal end region and a proximal end region, wherein the distal end region of the cannula is configured to be positioned in a left ventricle of a heart. Further, the cardiac pump system includes a first flow sensor coupled to the cannula or the catheter shaft, wherein the first flow sensor is configured to directly sense a first velocity of blood flowing adjacent to the first flow sensor.

### CROSS-REFERENCE TO RELATED APPLICATIONS

This application claims the benefit of priority under 35 U.S.C. § 119 of U.S. Provisional Application No. 63/499,107, filed Apr. 28, 2023, the entire disclosure of which is hereby incorporated by reference.

### TECHNICAL FIELD

The present disclosure relates to percutaneous circulatory support device systems. More specifically, the disclosure relates to percutaneous circulatory support devices that include one or more flow sensors.

### BACKGROUND

Percutaneous circulatory support devices such as blood pumps can provide transient cardiac support in patients whose heart function or cardiac output is compromised. Such devices may be delivered percutaneously from the femoral artery, retrograde through the descending aorta, over the aortic arch, through the ascending aorta across the aortic valve, and into the left ventricle. Some percutaneous circulatory support devices may include one or more flow sensors positioned thereon for directly measuring the flow of blood adjacent thereto. The direct measurement of blood flow may help derive the position of the support device within the heart, the cardiac output of the heart or other cardiac parameters. Accordingly, there is an ongoing need to provide circulatory support device systems including one or more flow sensors designed to provide cardiac information and/or other data related to a cardiac procedure. Circulatory support device systems including one or more flow sensors designed to provide cardiac information and/or other data related to a cardiac procedure are disclosed herein.

### SUMMARY

This disclosure provides design, material, manufacturing method, and use alternatives for medical devices and/or systems. An example cardiac pump system includes a catheter shaft having a proximal end region coupled to a handle and a distal end region coupled to a cardiac pump, wherein the cardiac pump includes an impeller housing, a cannula and an impeller, wherein the cannula includes a distal end region and a proximal end region, wherein the distal end region of the cannula is configured to be positioned in a left ventricle of a heart. Further, the cardiac pump system includes a first flow sensor coupled to the cannula or the catheter shaft, wherein the first flow sensor is configured to directly sense a first velocity of blood flowing adjacent to the first flow sensor.

Alternatively or additionally to any of the embodiments above, further comprising a console coupled to the handle, wherein the console includes a processor, and wherein the console is configured to receive a first signal from the first flow sensor.

Alternatively or additionally to any of the embodiments above, wherein the first signal corresponds to the first velocity of blood sensed by the first flow sensor.

Alternatively or additionally to any of the embodiments above, wherein the processor is configured to calculate a cardiac output of the heart based on the first velocity of blood sensed by the first flow sensor.

Alternatively or additionally to any of the embodiments above, wherein the first flow sensor is attached to an outer surface of the distal end region of the cannula.

Alternatively or additionally to any of the embodiments above, wherein the first flow sensor is embedded within a wall of the cannula.

Alternatively or additionally to any of the embodiments above, wherein the first flow sensor is coupled to the cannula, and wherein the cardiac pump system further includes a second flow sensor coupled to the catheter shaft.

Alternatively or additionally to any of the embodiments above, wherein the second flow sensor is configured to directly sense a second velocity of blood flowing adjacent to the second flow sensor, and wherein the console is configured to receive a second signal from the second flow sensor, and wherein the second signal corresponds to the second velocity of blood flowing adjacent to the second flow sensor.

Alternatively or additionally to any of the embodiments above, wherein the processor is configured to compare the first velocity of blood sensed by the first flow sensor to the second velocity of blood sensed by the second flow sensor.

Alternatively or additionally to any of the embodiments above, wherein the processor is configured to calculate a position of the cardiac pump based on a comparison of the first velocity of blood sensed by the first flow sensor to the second velocity of blood sensed by the second flow sensor.

Alternatively or additionally to any of the embodiments above, wherein the first flow sensor is positioned along the catheter shaft such that it is positioned distal of a subclavian artery when the distal end region of the cannula is positioned in the left ventricle.

Alternatively or additionally to any of the embodiments above, wherein the first flow sensor is positioned along the catheter shaft such that it is positioned proximal of a subclavian artery when the distal end region of the cannula is positioned in the left ventricle.

Alternatively or additionally to any of the embodiments above, wherein the first flow sensor is positioned along the catheter shaft such that it is positioned adjacent to and distal of a renal artery when the distal end region of the cannula is positioned in the left ventricle.

Alternatively or additionally to any of the embodiments above, wherein the first flow sensor is positioned along the catheter shaft such that it is positioned adjacent to and proximal of a renal artery when the distal end region of the cannula is positioned in the left ventricle.

Another example cardiac pump system includes a console including a processor and a cardiac pump device including a handle coupled to the console, a first catheter shaft having a proximal end region coupled to the handle and a distal end region coupled to a cardiac pump, an impeller and a cannula, wherein the cannula includes a proximal end region and a distal end region. Further, the cardiac pump system also includes a first flow sensor coupled to the proximal end region of the cannula, wherein the first flow sensor is positioned between the impeller and the distal end region of the cannula.

Alternatively or additionally to any of the embodiments above, wherein the first flow sensor is configured to directly sense a first velocity of blood flowing adjacent to the first flow sensor.

Alternatively or additionally to any of the embodiments above, wherein the processor is configured to calculate a cardiac output of the heart based on the first velocity of blood sensed by the first flow sensor.

Alternatively or additionally to any of the embodiments above, wherein the cardiac pump device further includes one or more blood inlets positioned on the distal end region of the cannula, and wherein the one or more blood inlets are configured to be positioned in a left ventricle of a heart.

Alternatively or additionally to any of the embodiments above, further comprising a second flow sensor coupled to the catheter shaft.

Another example method for positioning a cardiac pump system in a heart includes advancing a cardiac pump device adjacent to a left ventricle of the heart, the cardiac pump device including an impeller, a cannula including a proximal end region and a distal end region, and a first flow sensor coupled to the proximal end region of the cannula, wherein the first flow sensor is positioned between the impeller and the distal end region of the cannula. The method also includes positioning the distal end region of the cannula in the left ventricle and positioning the impeller in the ascending aorta of the heart.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

FIG. 1 depicts a percutaneous circulatory support system, including a circulatory support device and its relative position in a heart of a patient;

FIG. 2 is a schematic block diagram of a console management system;

FIG. 3 depicts a portion of the circulatory support system shown in FIG. 1 positioned in the heart of a patient;

FIG. 4 depicts a portion of a circulatory support system;

FIG. 5 is a cross-section taken along line 4-4 of FIG. 4;

FIG. 6 depicts an alternative embodiment of the cross-section taken along line 4-4 of FIG. 4;

FIG. 7 depicts an alternative embodiment of the cross-section taken along line 4-4 of FIG. 4;

FIG. 8 depicts a portion of a circulatory support system positioned in a body vessel;

FIG. 9 is a cross-section taken along line 8-8 of FIG. 8;

FIG. 10 depicts a portion of a circulatory support system positioned adjacent the renal arteries of a patient;

FIG. 11 depicts a portion of another circulatory support system positioned in the heart of a patient;

FIG. 12 depicts a portion of another circulatory support system positioned in the heart of a patient.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

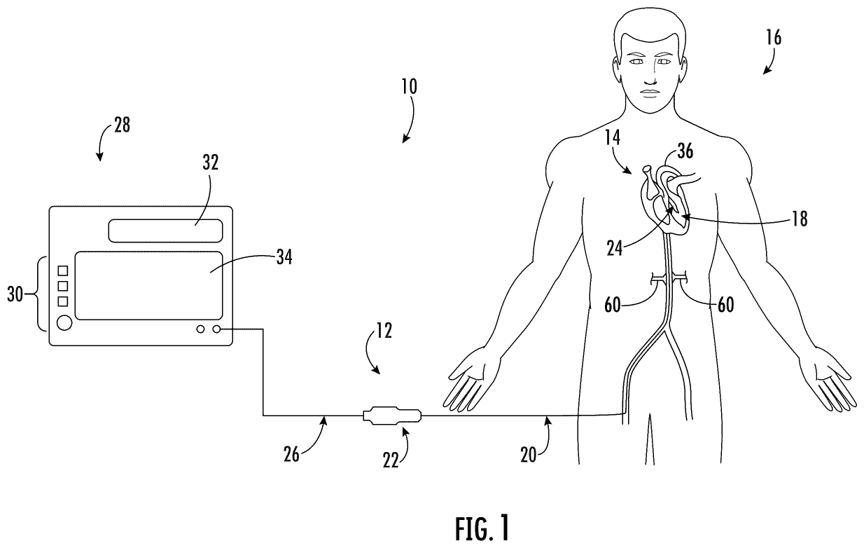

FIG. 1 illustrates an example percutaneous circulatory system 10 including a circulatory support device 12 positioned in the heart 14 of a patient 16. The circulatory support device 12 may include a flexible elongated catheter shaft 20 having a first end attached to a handle 22 and a second end attached to a blood pump 24. FIG. 1 illustrates the blood pump 24 positioned in the left ventricle 18 of the patient 16. The blood pump 24 may be delivered (e.g., tracked) to the ventricle 18 percutaneously over a guidewire. For example, the catheter shaft 20 and blood pump 24 may be tracked over a guidewire through the femoral artery, past the renal arteries 60 and the descending aorta, over the aortic arch, through the ascending aorta 37, past the aortic valve and into the left ventricle 18.

FIG. 1 further illustrates that the handle 22 may include a distal end region attached to the catheter shaft 20 and a proximal end region attached to an electrical power cable 26. The electrical power cable 26 may include a distal end region connected to a console 28. It can be appreciated that the handle 22 may include one or more actuators (e.g., buttons, levers, dials, switches, etc.) designed to permit a clinician to control various functions of the blood pump 24. For example, a clinician may be able to control the speed of the motor and/or an impeller located in the blood pump 24 via actuation of one or more actuators located on the handle 22.

Additionally, FIG. 1 illustrates that the console 28 may include one or more control knobs (e.g., buttons, knobs, dials, etc.) 30 and/or one or more displays. For example, FIG. 1 illustrates the console 28 may include a first display 32 and a second display 34. It can be appreciated that the console 28 may include more than two displays. Additionally, while FIG. 1 illustrates the first display 32 and the second display 34 integrated into the console 28, it is contemplated that the circulatory system 10 may be designed such that the first display 32, the second display 34 or both the first display 32 and the second display 34 are separate, distinct components of the circulatory system 10. In other words, the first display 32, the second display 34 or both the first display 32 and the second display 34 may be separate stand-alone displays, apart from the console 28. In some examples, the first display 32 and the second display 34 may get their respective data from separate sources.

In some examples, the second display 34 may be designed to attach to the console 28 and/or the first display 32. For example, the first display 32 may be integrated into the console 28 while the second display 34 may be configured to attach to portion of the console 28. In yet other examples, both the first display 32 and the second display 34 may be a separate stand-alone display whereby the second display 34 may be configured to attach to the first display 32, or wherein the first display 32 may be configured to attach to the second display 34.

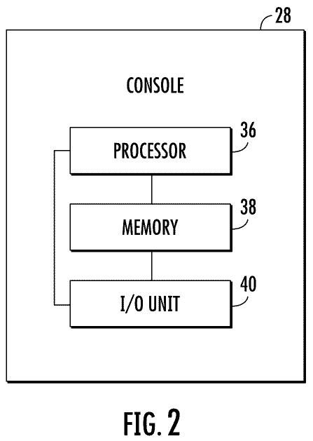

FIG. 2 illustrates that the console 28 may include, among other suitable components, one or more processors 36, memory 38, and an I/O unit 40. The processor 36 of the console 28 may include a single processor or more than one processor (e.g., a first processor 36 providing data/instructions to the first display 32 and a second processor 36 providing data instructions to a second display 34) working individually or with one another. The processor 36 may be configured to execute instructions, including instructions that may be loaded into the memory 38 and/or other suitable memory. Example processor components may include, but are not limited to, microprocessors, microcontrollers, multi-core processors, graphical processing units, digital signal processors, application specific integrated circuits (ASICs), field programmable gate arrays (FPGAs), discrete circuitry, and/or other suitable types of data processing devices. In some examples, the processor 36 of the console may be configured to execute program instructions. Program instructions may include, for example, firmware, microcode or application code that is executed by the processor 36, a microprocessor and/or microcontroller. The one or more processors 36 may be configured to each manage different functions. They may also be configured to concurrently perform the same functions (e.g., redundant system). Further yet, they may be configured such that a first processor 36 performs a given function and second processor 36 checks the result of the function of the first processor 36 for correctness (e.g., command-monitor system).

In some examples, the first display 32 may be controlled primarily by the console's firmware control instructions and, therefore, may require relatively little processing power, relatively few instructions and very simple communication between the processor 36 and the display 32, compared to the second display 34 (e.g., a touch screen display 34), which may be controlled primarily by an embedded computer with a flexible and relatively complex communication protocol.

The memory 38 of the console 28 may include a single memory component or more than one memory component each working individually or with one another. Example types of memory may include random access memory (RAM), EEPROM, FLASH, suitable volatile storage devices, suitable non-volatile storage devices, persistent memory (e.g., read only memory (ROM), hard drive, Flash memory, optical disc memory, and/or other suitable persistent memory) and/or other suitable types of memory. The memory 38 may be or may include a non-transitory computer readable medium.

The I/O units 40 of the console 28 may include a single I/O component or more than one I/O component each working individually or with one another. Example I/O units 40 may be any type of communication port configured to communicate with other components of the circulatory system 10. Example types of I/O units 45 may include wired ports, wireless ports, radio frequency (RF) ports, Low-Energy Bluetooth ports, Bluetooth ports, Near-Field Communication (NFC) ports, HDMI ports, Wi-Fi ports, Ethernet ports, VGA ports, serial ports, parallel ports, component video ports, S-video ports, composite audio/video ports, DVI ports, USB ports, optical ports, and/or other suitable ports.

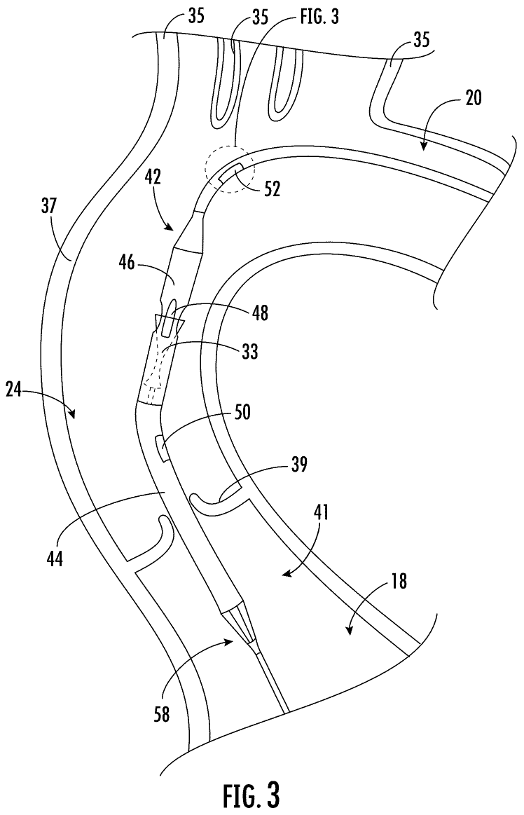

FIG. 3 illustrates the blood pump 24 of the percutaneous circulatory system 10 extending from the ascending aorta 37 to the left ventricle 18 of a patient 16. The blood pump 24 may include a cannula 44 having a proximal end attached to a distal end of an impeller housing 46. A proximal end 42 of the impeller housing 46 may be attached to a distal end of the catheter shaft 20. FIG. 3 illustrates that, in some examples, the blood pump 24 may be positioned within the heart 14 such that the cannula 44 passes through the aortic valve 39, whereby a distal end region 41 of the cannula 44 may be positioned within the left ventricle 18. As discussed herein, the blood pump 24 may be tracked over a guidewire to its position illustrated in FIG. 3.

FIG. 3 further illustrates that the shaft 20 of the circulatory support device 12 may include one or more blood inlets 58 located on a distal end region 41 of the cannula 44, and one or more blood outlets 48 positioned along the impeller housing 46. In some examples, the blood pump 24 may be positioned within the heart 14 such that the one or more blood inlets 58 positioned along the distal end region 41 of the cannula 44 may be positioned in the left ventricle 18 and the one or more blood outlets 48 located along the impeller housing 46 may be positioned in the ascending aorta 37.

Additionally, the blood pump 24 may include an electrically powered motor that drives rotation of the impeller 33 which may be positioned within the impeller housing 46. In some examples, the motor may power the rotation of the impeller 33 via electromagnetic induction. The spinning impeller 33 may draw blood from the left ventricle 18 (via the one or more blood inlets 58 located on a distal region of the cannula 44) into the ascending aorta 37 (via the one or more blood outlets 48 located along the impeller housing 46). In other words, an electrically powered motor drives the impeller 33 to pump blood from the left ventricle 18 through the aortic valve 39 and into the ascending aorta 37.

Additionally, the circulatory support device 12 may include one or more sensors coupled to the cannula 44, the impeller housing 46 and/or the catheter shaft 20. The one or more sensors coupled to the cannula 44, the impeller housing 46 and/or the catheter shaft 20 may be designed to monitor blood pressures (e.g., arterial pressure, venous pressure), blood velocity, or other relevant cardiac parameters. Additionally, the one or more sensors of the circulatory support device 12 coupled to the cannula 44, the impeller housing 46 and/or the catheter shaft 20 may be designed to monitor other parameters of the circulatory system 10, the circulatory support device 12 and/or the patient 16.

FIG. 3 illustrates that, in some examples, the percutaneous circulatory system 10 may include a sensor 50 (e.g., flow sensor, position sensor, etc.) positioned along a distal end region of the cannula 44. For example, in which the sensor 50 is a flow sensor, the flow sensor 50 may be designed to sense the flowrate of blood (e.g., blood velocity) adjacent to the flow sensor 50. For example, the flow sensor 50 may be designed to directly measure the velocity of blood passing from the left ventricle 18, through the aortic valve 39 and into the ascending aorta 37. Any of the example flow sensors described herein may include an optical sensor, ultrasound sensor, electromagnetic sensor, thermoconvection sensor, etc. Additionally, for examples in which the sensor 50 is a position sensor, the sensor 50 may be able to determine the positione of the blood pump 24 relative to the left ventricle 18 and/or the aorta 37 based on a sensed measurement of the blood velocity passing adjacent to the sensor 50.

FIG. 3 further illustrates that, in some examples, the percutaneous circulatory system 10 may include a flow sensor 52 positioned along a distal end region of the catheter shaft 20. In some examples, the flow sensor 52 may be positioned superior to the subclavian arteries 35. In other examples, the flow sensor 52 may be positioned inferior to the subclavian arteries 35. In yet other examples, the flow sensor 52 may be positioned about 1 cm to 10 cm from the proximal end of the impeller housing 46, or about 2 cm to 9 cm from the proximal end of the impeller housing 46, about 3 cm to 7 cm from the proximal end of the impeller housing 46, or about 4 cm to 6 cm from the proximal end of the impeller housing 46, or about 5 cm from the proximal end of the impeller housing 46. The flow sensor 52 may be designed to sense the flowrate of blood (e.g., blood velocity) adjacent to the flow sensor 52. For example, the flow sensor 52 may be designed to directly measure the velocity of blood passing from the left ventricle 18, through the aortic valve 39 and into the ascending aorta 37. Any of the example flow sensors described herein may include an optical sensor, ultrasound sensor, electromagnetic sensor, thermoconvection sensor, etc.

Additionally, it can be appreciated that, in some examples, the flow sensor 52 may be positioned within the descending aorta, superior to the celiac trunk, inferior to the celiac trunk, superior to the renal arteries and/or inferior to renal arteries.

In some instances, the position of the blood pump 24 (including the position of the blood inlets 58, the blood outlets 48 and/or the impeller 33) may be determined by comparing a blood velocity measurement taken at the flow sensor 50 with a blood velocity measurement taken at the flow sensor 52. For example, the processing components 36 of the percutaneous circulatory system 10 may include an algorithm designed to receive and compare the blood velocity data sent from the flow sensor 50 with the blood velocity data from the flow sensor 52. It can be appreciated that the velocity of blood passing by the flow sensor 50 may be less than the velocity of blood passing by the flow sensor 52 because the velocity of blood passing by the flow sensor 52 may be increased (e.g., assisted) by the impeller 33. In other words, the velocity of blood exiting the blood outlets 48 may be greater than the blood passing by the flow sensor 50 and the blood entering the blood inlets 58. Further, knowing the distance between the flow sensor 50 and the flow sensor 52, the processing components 36 may be able to calculate the relative position of the impeller housing (positioned between the flow sensor 50 and the flow sensor 52) by comparing the blood velocity measurement taken at the flow sensor 50 with the blood velocity measurement taken at the flow sensor 52.

Additionally, it can be appreciated that, for any of the flow sensors described herein (e.g., flow sensor 50, flow sensor 52, etc.), the blood velocity directly measured by a given flow sensor (e.g., flow sensor 50, flow sensor 52, etc.) may be utilized to calculate a measurement of cardiac output. Cardiac output may be defined as the amount of blood pumped per unit of time (e.g., the volumetric flowrate of blood within the body). Cardiac output may be calculated by multiplying the stroke volume (e.g., the volume of blood exiting the left ventricle per stroke) by the heart rate (e.g., the number of strokes of the left ventricle per unit time).

It can be further appreciated that, in some examples, the processing components 36 of the percutaneous circulatory system 10 may include an algorithm designed to receive and compare the blood velocity data sent from any of the flow sensors described herein (e.g., flow sensor 50, flow sensor 52, etc.) and use it to calculate the cardiac output of the heart 14. Further, any of the flow sensors described herein (e.g., the flow sensor 50 and the flow sensor 52) may be coupled to and/or incorporate an impedance sensor, whereby the impedance sensor may be utilized to determine (e.g., calculate) the diameter of a body vessel (e.g., the ascending aorta, descending aorta, etc.) adjacent to a flow sensor (e.g., the flow sensor 50 and the flow sensor 52). It can be appreciated that the diameter of a body vessel (e.g., the ascending aorta, descending aorta, etc.) may be utilized by the processing components 36 to calculate the cardiac output of the heart 14.

Further yet, one or more components of the percutaneous circulatory system 10 may be coupled to an ultrasound system capable of utilizing ultrasound to determine the diameter of a body vessel (e.g., the ascending aorta, descending aorta, etc.) adjacent to a flow sensor (e.g., the flow sensor 50 and the flow sensor 52). The ultrasound system may communicate with the processing components 36 of the percutaneous circulatory system 10. Accordingly, the processing components 36 of the percutaneous circulatory system 10 may include an algorithm capable of receiving data from the ultrasound system corresponding to the diameter of a body vessel (e.g., the ascending aorta, descending aorta, etc.) adjacent to a flow sensor (e.g., the flow sensor 50 and the flow sensor 52). Further, the data received from the ultrasound system may be utilized by the processing components 36 of the percutaneous circulatory system 10 to calculate the cardiac output of the heart 14.

In yet another example, the percutaneous circulatory system 10 may include a conductance catheter configured to measure the conductance of blood between two equally spaced electrodes positioned on the conductance catheter, whereby the volume of blood may be calculated based on the conductance reading. Further, knowing the volume of blood in a blood vessel at an instance in time may be used to estimate the diameter of the vessel adjacent to the electrodes. Accordingly, the processing components 36 of the percutaneous circulatory system 10 may include an algorithm capable of receiving data from the conductance catheter corresponding to the diameter of a body vessel (e.g., the ascending aorta, descending aorta, etc.). Further, the data received from the conductance catheter may be utilized by the processing components 36 of the percutaneous circulatory system 10 to calculate the cardiac output of the heart 14.

It can be appreciated that any of the flow sensors described herein (e.g., flow sensor 50, flow sensor 52, etc.) may send signals to the console 28 and/or the processing components 26 via a wireless connection (e.g., a Bluetooth connection). In other examples, any of the flow sensors described herein (e.g., flow sensor 50, flow sensor 52, etc.) may be hardwired to the console 28 and/or the processing components 26.

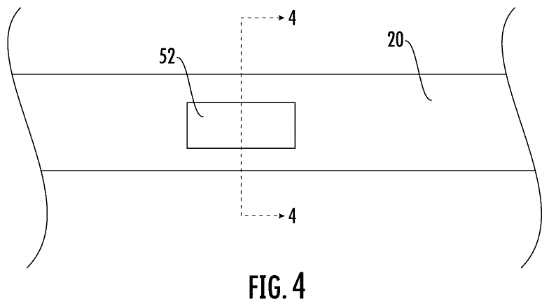

FIG. 4 illustrates an example flow sensor (e.g., the flow sensor 52) attached to the catheter shaft 20 of the circulatory support device 12. FIG. 4 illustrates that the flow sensor 52 may be sized and shaped to permit the blood to flow across the surface area thereof without substantially impeding or disturbing the velocity and/or fluid dynamics of the blood flow. For example, FIG. 4 illustrates the flow sensor 52 having a generally rectangular shape and extending along the longitudinal axis of the catheter shaft 20. However, it can be appreciated that the sensor 52 may include any shape, including a circular, ovular, square, triangular, polygonal, star-shaped, or any combinations thereof.

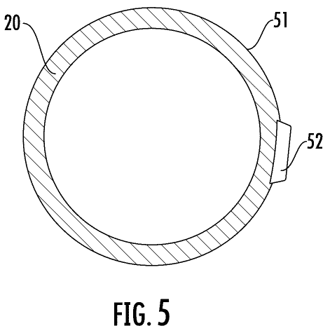

FIG. 5 illustrates a cross-sectional view taken along line 4-4 of FIG. 4. FIG. 5 illustrates that the sensor 52 may be attached to an outer surface 51 of the catheter shaft 20. For example, FIG. 5 illustrates that, in some examples, the sensor 52 may be a separate component from the catheter shaft 20, whereby the sensor 52 is attached (e.g., via adhesive, etc.) directly to the outside of the catheter shaft 20.

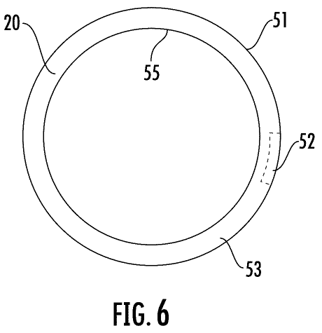

However, FIG. 6 illustrates an alternative example in which the example sensor 52 is embedded within the wall 53 of the catheter shaft 20. In some examples, the sensor 52 may be positioned within the wall 53 of the catheter shaft 20 during an extrusion process of the catheter shaft 20. Further, in some examples, the sensor 52 may include a first surface which is substantially flush with the outer surface 51 of the catheter shaft. In other examples, the sensor 52 may be positioned (e.g., embedded) between the inner surface 55 and the outer surface 51 of the catheter shaft 20.

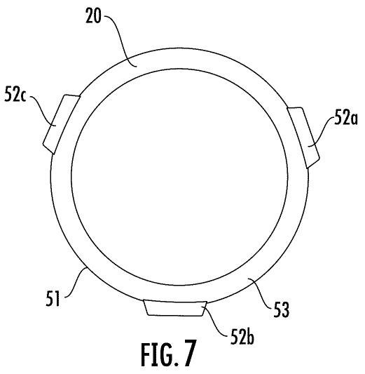

FIG. 7 illustrates that, in some examples, more than one sensor may be positioned along the outer surface of the catheter shaft 20. For example, FIG. 7 illustrates three sensors 52 positioned along the outer surface 51 of the catheter shaft 20. It can be appreciated that the sensors 52 may be circumferentially spaced equidistant from one another around the outer surface 51 of the catheter shaft. For example, the sensors 52a, 52b, 52c shown in FIG. 7 may be circumferentially spaced substantially 180 degrees from one another. It can be further appreciated that the circulatory support device 12 may include 1, 2, 3, 4, 5, 6, 7, 8, 9, 10 or more sensors positioned adjacent one another at a given longitudinal location along the blood pump (e.g., the cannula 44, the impeller housing 46) and/or the catheter shaft 20. At a given longitudinal location, the sensors may be circumferentially spaced substantially equidistant from one another or they may not be spaced equidistant from one another. Further, one or more of any of the sensors described herein may be either attached directly to an outer surface of a portion of the blood pump (e.g., the cannula 44, the impeller housing 46) and/or the catheter shaft 20 or the one or more of any of the sensors described herein may be embedded within the wall of a portion of the blood pump (e.g., the cannula 44, the impeller housing 46) and/or the catheter shaft 20.

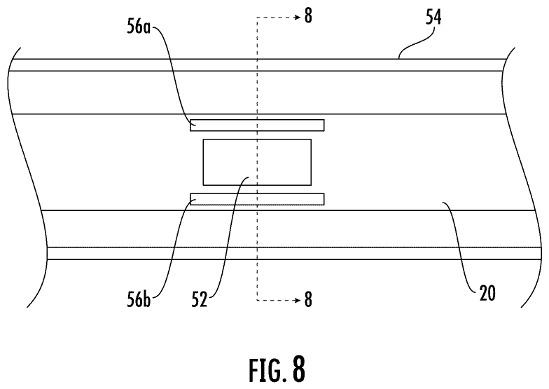

FIG. 8 illustrates an example in which a component of the circulatory support device 12 (e.g., the cannula 44, the impeller housing 46 and/or the catheter shaft 20) may include one or more features which are designed to space a flow sensor away from the wall of a body vessel 54 (e.g., the inner wall of the ascending aorta 37). For example, FIG. 8 illustrates the catheter shaft 20 may include a first spine 56a positioned adjacent to the sensor 52 and a second spine 56b positioned adjacent to the sensor 52. It can be appreciated from FIG. 8 that the sensor 52 may be positioned between the first spine 56a and the second spine 56b. As viewed in FIG. 8, the first spine 56a may be positioned above the sensor 52 and the second spine 56b may be positioned below the sensor 52.

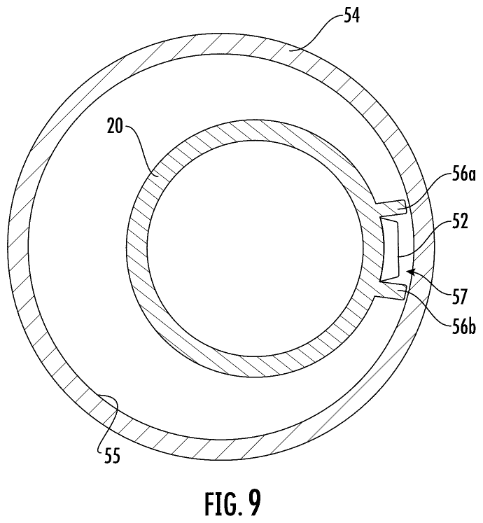

FIG. 9 illustrates a cross-sectional view taken along line 8-8 of FIG. 8. FIG. 9 illustrates an example in which the catheter shaft 20 has shifted toward the inner surface 55 of the body vessel 54. For example, FIG. 9 may resemble a situation during a medical operation in which the catheter shaft 20 has pushed up against the inner surface of the ascending aorta of the heart. FIG. 9 further illustrates that the first spine 56a and the second spine 56b may contact the inner surface 55 of the body vessel 54, thereby creating a gap 57 between the flow sensor 52 and the inner surface 55 of the body vessel 54. It can be appreciated that the gap 57 may permit blood to flow across the flow sensor 52, thereby allowing the flow sensor to directly measure the blood velocity adjacent to the flow sensor 52 despite the catheter shaft 20 having been shifted toward the inner surface 55 of the body vessel 54.

It can further be appreciated that a component of the circulatory support device 12 (e.g., the cannula 44, the impeller housing 46 and/or the catheter shaft 20) may include features other than or in addition to the spine 56a and/or the spine 56b designed to create a space or gap between a flow sensor (e.g., the flow sensor 50, the flow sensor 52, etc.), thereby allowing blood to flow past the sensor 52 despite the component having been shifted toward the inner surface of a body vessel. For example, a component of the circulatory support device 12 (e.g., the cannula 44, the impeller housing 46 and/or the catheter shaft 20) may include one or more projections, bumps, protrusions, spikes, ridges, rims, steps, etc. positioned adjacent to a flow sensors to space the flow sensor away from an inner surface of the vessel wall.

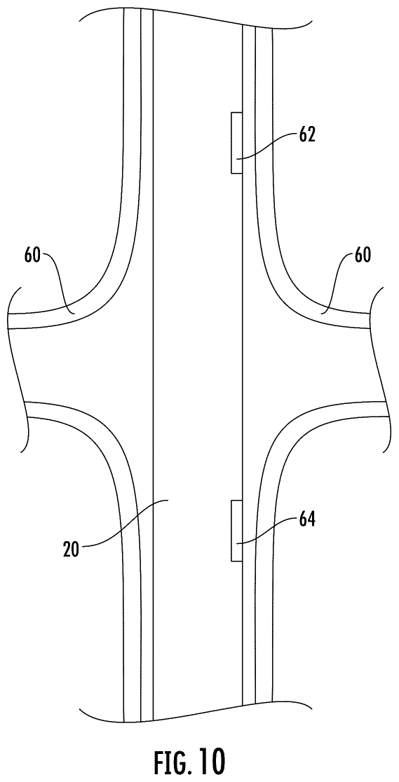

FIG. 10 illustrates that the circulatory support device 12 may include a flow sensor (similar in form and function to any of the flow sensors described herein) positioned adjacent to the renal arteries 60. For example, FIG. 10 illustrates that a flow sensor 62 may be attached to the catheter shaft 20 and positioned superior of the renal arteries 60. FIG. 10 illustrates that a flow sensor 64 may be attached to the catheter shaft 20 and positioned inferior of the renal arteries 60. The flow sensors 62, 64 may be designed and perform similarly to any of the example flow sensors (e.g., sensors 50, 52) described herein. For example, the flow sensors 62, 64 may be designed to directly measure the velocity of blood passing thereby. Further, the velocity data collected by the sensors 62, 64 may be utilized by the processing components 36 to calculate the positioned of the circulatory support device 12 within the body vessel and/or calculate the cardiac output of the heart 14, as described herein.

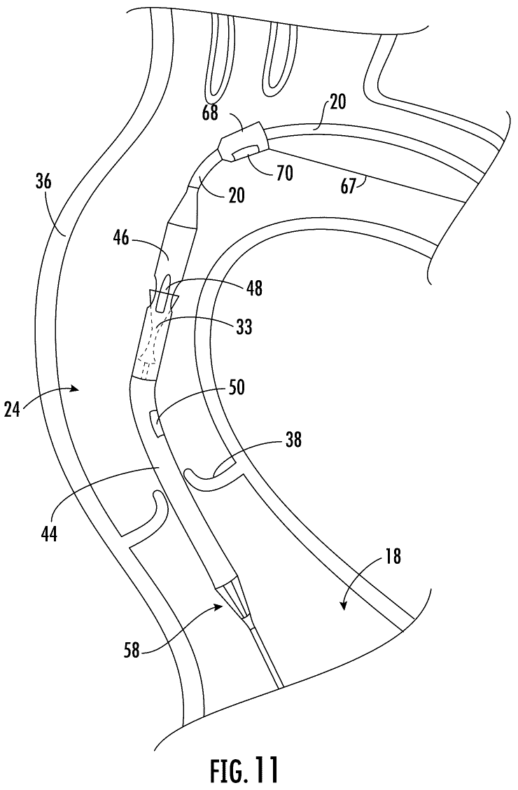

FIG. 11 illustrates that, in some instances, the percutaneous circulatory system 10 may further include a flow-sensing component which is designed to track over and along the catheter shaft 20. For example, FIG. 11 illustrates that the percutaneous circulatory system 10 may include a secondary sensor assembly 66 which includes a sensor housing 68 and a flow sensor 70 positioned along a distal end region thereof. It can be appreciated that the sensor housing 68 may include a lumen sized to permit the catheter shaft 20 to pass therethrough. Accordingly, the catheter assembly 66 (including the sensor housing 68 and the flow sensor 70) may translate (e.g., slide) relative to the catheter shaft 20 and/or the blood pump 24 (including the cannula 44 and the impeller housing 46), thereby permitting the flow sensor 70 to be positioned at position within body vessel (e.g., aorta) relative to the flow sensor 50, the flow sensor 52 (which may or may not be included in the example system shown in FIG. 11), the impeller 33, the impeller housing 46, the cannula 44, the blood inlets 58, the blood outlets 48 or any other component of the system 10.

In some examples, the catheter assembly 66 may further include a push-wire 67 coupled to the catheter shaft 68. In some examples, the push-wire 67 may be attached to an outer surface of the of the sensor housing 68. The push-wire 67 may be designed to aid in the tracking of the sensor housing 68 over the catheter shaft 20. For example, the push-wire 67 may provide additional strength to the sensor housing 68 which helps push the sensor housing 68 along the catheter shaft 20 to a desired position within the body vessel.

Additionally, it can be appreciated that the flow sensor 70 may be designed and perform similarly to any of the example flow sensors (e.g., sensors 50, 52, 62, 64) described herein. For example, the flow sensor 70 may be designed to directly measure the velocity of blood passing thereby. Further, the velocity data collected by the sensor 70 may be utilized by the processing components 36 to calculate the position of the circulatory support device 12 within the body vessel and/or calculate the cardiac output of the heart 14, as described herein.

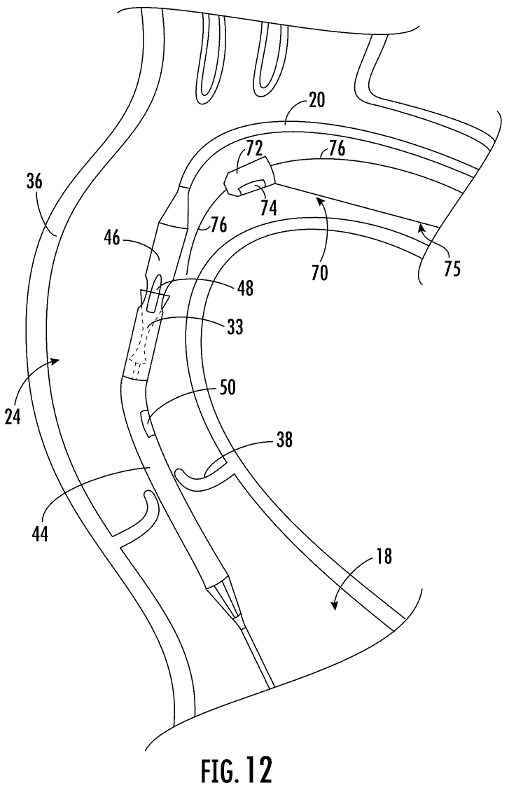

FIG. 12 illustrates that, in some instances, the percutaneous circulatory system 10 may further include a flow-sensing assembly which is separate from the components of the circulatory support device 12. For example, FIG. 12 illustrates that the percutaneous circulatory system 10 may include a separate sensing assembly 70 which includes a sensor housing 72 and a flow sensor 74 positioned along a distal end region thereof. It can be appreciated that the sensor housing 72 may include a lumen sized to permit a guidewire 76 (separate from a guidewire utilized to position the blood pump 24) to pass therethrough. Accordingly, the sensing assembly 70 (including the sensor housing 72 and the flow sensor 74) may translate (e.g., slide) may be positioned alongside and relative to the guidewire 76 and/or the blood pump 24 (including the cannula 44 and the impeller housing 46), thereby permitting the flow sensor 74 to be positioned at position within body vessel (e.g., aorta) relative to the flow sensor 50, the flow sensor 52 (which may or may not be included in the example system shown in FIG. 12), the impeller 33, the impeller housing 46, the cannula 44, the blood inlets 58, the blood outlets 48 or any other component of the system 10.

In some examples, the catheter assembly 70 may further include a push-wire 75 coupled to the sensor housing 72. In some examples, the push-wire 75 may be attached to an outer surface of the of the sensor housing 72. The push-wire 75 may be designed to aid in the tracking of the sensor housing 72 over the guidewire 76. For example, the push-wire 75 may provide additional column strength to the sensor housing 72 which helps push the sensor housing 72 along the guidewire 76 to a desired position within the body vessel.

Additionally, it can be appreciated that the flow sensor 74 may be designed and perform similarly to any of the example flow sensors (e.g., sensors 50, 52, 62, 64. 70) described herein. For example, the flow sensor 74 may be designed to directly measure the velocity of blood passing thereby. Further, the velocity data collected by the sensor 74 may be utilized by the processing components 36 to calculate the position of the circulatory support device 12 within the body vessel and/or calculate the cardiac output of the heart 14, as described herein.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

1. A cardiac pump system, comprising:
a catheter shaft having a proximal end region coupled to a handle and a distal end region coupled to a cardiac pump, wherein the cardiac pump includes an impeller housing, a cannula and an impeller, wherein the cannula includes a distal end region and a proximal end region, wherein the distal end region of the cannula is configured to be positioned in a left ventricle of a heart; and
a first flow sensor coupled to the cannula or the catheter shaft, wherein the first flow sensor is configured to directly sense a first velocity of blood flowing adjacent to the first flow sensor.

2. The cardiac pump system of claim 1, further comprising a console coupled to the handle, wherein the console includes a processor, and wherein the console is configured to receive a first signal from the first flow sensor.

3. The cardiac pump system of claim 2, wherein the first signal corresponds to the first velocity of blood sensed by the first flow sensor.

4. The cardiac pump system of claim 3, wherein the processor is configured to calculate a cardiac output of the heart based on the first velocity of blood sensed by the first flow sensor.

5. The cardiac pump system of claim 1, wherein the first flow sensor is attached to an outer surface of the distal end region of the cannula.

6. The cardiac pump system of claim 1, wherein the first flow sensor is embedded within a wall of the cannula.

7. The cardiac pump system of claim 2, wherein the first flow sensor is coupled to the cannula, and wherein the cardiac pump system further includes a second flow sensor coupled to the catheter shaft.

8. The cardiac pump system of claim 7, wherein the second flow sensor is configured to directly sense a second velocity of blood flowing adjacent to the second flow sensor, and wherein the console is configured to receive a second signal from the second flow sensor, and wherein the second signal corresponds to the second velocity of blood flowing adjacent to the second flow sensor.

9. The cardiac pump system of claim 8, wherein the processor is configured to compare the first velocity of blood sensed by the first flow sensor to the second velocity of blood sensed by the second flow sensor.

10. The cardiac pump system of claim 9, wherein the processor is configured to calculate a position of the cardiac pump based on a comparison of the first velocity of blood sensed by the first flow sensor to the second velocity of blood sensed by the second flow sensor.

11. The cardiac pump system of claim 1, wherein the first flow sensor is positioned along the catheter shaft such that it is positioned distal of a subclavian artery when the distal end region of the cannula is positioned in the left ventricle.

12. The cardiac pump system of claim 1, wherein the first flow sensor is positioned along the catheter shaft such that it is positioned proximal of a subclavian artery when the distal end region of the cannula is positioned in the left ventricle.

13. The cardiac pump system of claim 1, wherein the first flow sensor is positioned along the catheter shaft such that it is positioned adjacent to and distal of a renal artery when the distal end region of the cannula is positioned in the left ventricle.

14. The cardiac pump system of claim 1, wherein the first flow sensor is positioned along the catheter shaft such that it is positioned adjacent to and proximal of a renal artery when the distal end region of the cannula is positioned in the left ventricle.

15. A cardiac pump system, comprising:
a console including a processor;
a cardiac pump device including a handle coupled to the console, a first catheter shaft having a proximal end region coupled to the handle and a distal end region coupled to a cardiac pump, an impeller and a cannula, wherein the cannula includes a proximal end region and a distal end region; and
a first flow sensor coupled to the proximal end region of the cannula, wherein the first flow sensor is positioned between the impeller and the distal end region of the cannula.

16. The cardiac pump system of claim 15, wherein the first flow sensor is configured to directly sense a first velocity of blood flowing adjacent to the first flow sensor.

17. The cardiac pump system of claim 16, wherein the processor is configured to calculate a cardiac output of the heart based on the first velocity of blood sensed by the first flow sensor.

18. The cardiac pump system of claim 17, wherein the cardiac pump device further includes one or more blood inlets positioned on the distal end region of the cannula, and wherein the one or more blood inlets are configured to be positioned in a left ventricle of a heart.

19. The cardiac pump system of claim 15, further comprising a second flow sensor coupled to the catheter shaft.

20. A method for positioning a cardiac pump system in a heart, the method comprising:
advancing a cardiac pump device adjacent to a left ventricle of the heart, the cardiac pump device including:
an impeller, a cannula including a proximal end region and a distal end region, and a first flow sensor coupled to the proximal end region of the cannula, wherein the first flow sensor is positioned between the impeller and the distal end region of the cannula;
positioning the distal end region of the cannula in the left ventricle; and
positioning the impeller in the ascending aorta of the heart.
